(19) United States
(12) Patent Application Publication
(10) Pub. No.: US 2023/0355950 A1
(43) Pub. Date: Nov. 9, 2023

(54) MEDICAL PORT PROTECTION ADAPTERS

(71) Applicant: Thomas Jefferson University, Philadelphia, PA (US)

(72) Inventors: Joseph Anthony Leone, Eagleville, PA (US); Jean Rex Geary, Newtown Square, PA (US); Lauren Renee Huggler, Philadelphia, PA (US); Anjali Jigisha Patel, Philadelphia, PA (US); Heli Amul Patel, Edison, NJ (US); Stephanie Francis Szymanski, Dublin, OH (US)

(21) Appl. No.: 18/029,422

(22) PCT Filed: Oct. 12, 2021
(86) PCT No.: PCT/US2021/071819

§ 371 Date: Mar. 30, 2023

Related U.S. Application Data
(60) Provisional application No. 63/091,079, filed on Oct. 13, 2020.

(51) Int. Cl. A61M 39/20

(52) U.S. Cl. CPC A61M 39/20; A61M 2205/0216; A61M 2205/0227

(57) ABSTRACT

Medical port protection adapters are described herein. In one aspect, a contamination guard can include a proximal collar adapted and configured to engage with a medical port; an elastomeric medial region coupled to the proximal collar; and a distal collar coupled to the proximal collar by the elastomeric medial region; where the elastomeric medial region is adapted and configured to: bias the distal collar to extend at least partially over a distal end of the port when in an unloaded position; and allow the distal collar to resiliently travel proximally when loaded to permit engagement with the distal end of the medical port.

### CROSS-REFERENCE TO RELATED APPLICATION

This application claims the benefit of priority under 35 U.S.C. § 119(e) to U.S. Provisional Patent Application Ser. No. 63/091,079, filed Oct. 13, 2020. The entire content of this application is hereby incorporated by reference herein.

### BACKGROUND OF THE INVENTION

Medical infusion can administer medication through a sterile catheter inserted into a patient's vein and secured. Medical infusion equipment, however, can be susceptible to contamination, particularly the male and female connectors of the infusion equipment. This can potentially lead to infections.

### SUMMARY

Medical port protection adapters are described herein. In one aspect, a contamination guard can include a proximal collar adapted and configured to engage with a medical port; an elastomeric medial region coupled to the proximal collar; and a distal collar coupled to the proximal collar by the elastomeric medial region; where the elastomeric medial region is adapted and configured to: bias the distal collar to extend at least partially over a distal end of the port when in an unloaded position; and allow the distal collar to resiliently travel proximally when loaded to permit engagement with the distal end of the medical port.

This aspect can include a variety of embodiments. In one embodiment, the proximal collar and the distal collar are annular. In another embodiment, the elastomeric medical region includes a plurality of legs. In some cases, the plurality of legs are axial, trussed, or helical.

In another embodiment, the distal collar is elastomeric. In another embodiment, the elastomeric medial region is fabricated from the group including: silicone and Methyl Methacrylate Acrylonitrile Butadiene Styrene (MABS).

In another embodiment, the contamination guard can further include a covering seal adapted and configured to be detachably coupled to the distal collar and across an aperture defined by the distal collar.

In another embodiment, the proximal collar defines a set of threads and grooves, where the set of threads and grooves is adapted and configured to couple with another set of threads and grooves defined by medical port. In another embodiment, the proximal collar is permanently coupled to the medical port.

In another embodiment, the contamination guard can further include an antimicrobial composition disposed on a surface of, or within a composition of, the proximal collar, the distal collar, the elastomeric medial region, or a combination thereof.

In another embodiment, the contamination guard can further include an indicator coating disposed on a surface of the proximal collar, the distal collar, the elastomeric medial region, or a combination thereof. In some cases, the indicator coating provides an indication of bacterial contamination, a pH change, an oxidation or material breakdown, or a combination thereof.

In another embodiment, the contamination guard can further include a sheet of elastomeric material covering a surface of a portion of the proximal collar, the distal collar, the elastomeric medial region, or a combination thereof.

In another embodiment, the elastomeric medial region is further adapted and configured to allow the distal collar to travel distally to a neutral position when unloaded.

In another embodiment, the distal end defines a set of ridges or flanges extending outwardly away from the distal end.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a side view of a female coupling adapter according to an embodiment of the claimed invention.

FIG. 2 depicts a proximal perspective view of a female coupling adapter according to an embodiment of the claimed invention.

FIG. 3 depicts a side perspective view of a male coupling adapter according to an embodiment of the claimed invention.

FIG. 4 depicts a side perspective view of male and female coupling adapters coupled to respective male and female connectors, according to embodiments of the claimed invention.

FIG. 5 depicts a side perspective of male and female coupling adapters coupled to respective male and female connectors, where the male and female connectors are coupled to each other, according to embodiments of the claimed invention.

FIG. 6 depicts a side perspective of a coupling adapter having a covering seal disposed across the defined top orifice according to an embodiment of the claimed invention.

FIG. 7 depicts corresponding male and female coupling adapters, each defining a conical shape, according to an embodiment of the claimed invention.

FIGS. 8 and 9 depicts corresponding male and female coupling adapters, each defining a flange extending outwardly from the respective distal collars.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

The disclosure described herein relate to coupling adapters for Luer taper connectors. A protection adapter can be coupled to a Luer taper connector or other medical connector such as a threaded fitting. The adapter can protect the connector from physically contacting other objects, which can decrease the potential of contamination for the connector. The adapter can also couple to another protection adapter that is coupled to the corresponding matching connector. For example, a protection adapter coupled to a male connector can couple to another protection adapter coupled to a female connector. Thus, not only are the individual connectors protected from physical contamination, but also the mated assembly is protected as well.

### Female Coupling Adapter

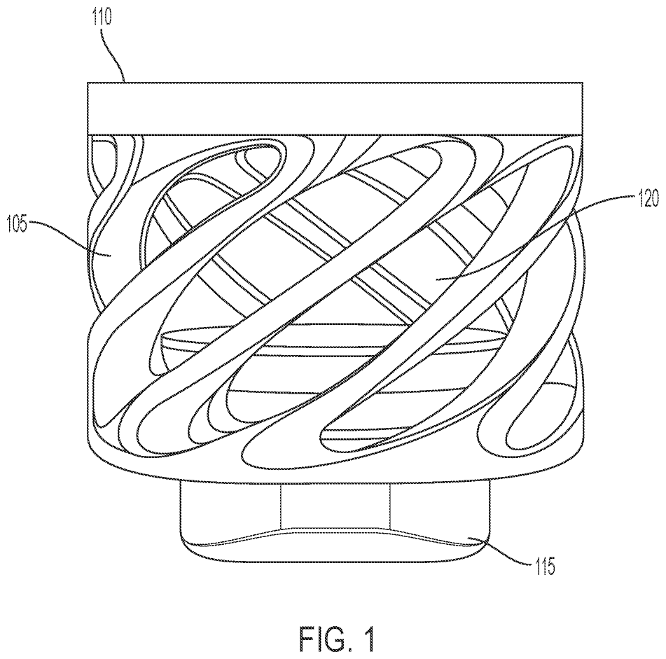

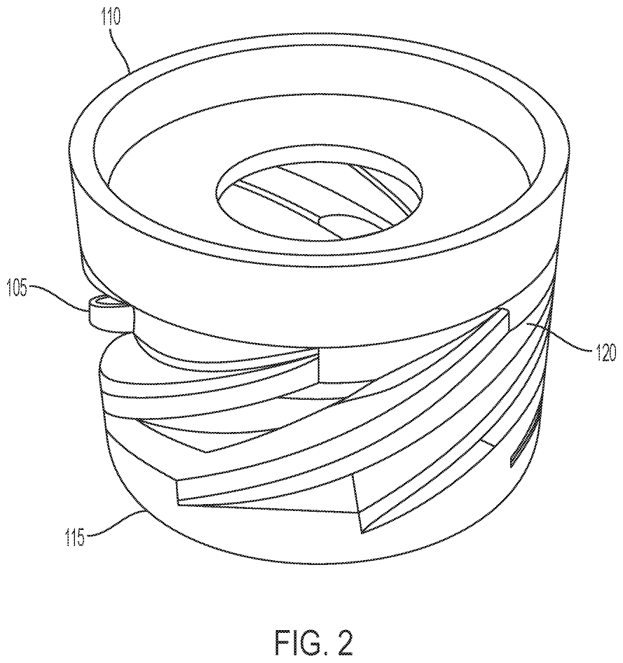

FIG. 1 depicts a female coupling adapter or contamination guard, according to an embodiment of the claimed invention. The adapter can include a hollow structure configured for encompassing a female medical connector. The adapter can include a set of sidewalls (e.g., a medial region) 105. In some cases, the adapter can be of a cylindrical shape, but the adapter can be other shapes as well (rectangle tube, helix, and the like). The sidewalls 105 can terminate to form a ridge (e.g., a distal collar) 110 or top wall on one end of the adapter. The ridge 110 can be configured to couple to an end of a corresponding male coupling adapter described in more detail below. For example, the ridge 110 can define a top orifice for accommodating a coupling between a male connector to a corresponding female connector. The sidewalls 105 can also couple to a bottom wall or distal collar 115 on another end of the adapter. The bottom wall 115 can define an orifice, which can be configured for accommodating the disposition of a female connector.

The female coupling adapter can be configured for housing or accommodating (e.g., within the hollow structure or defined cavity of the adapter) different female connectors or medical ports of varying widths and/or lengths, including universal female connectors. For example, the bottom wall 115 can be adapted and configured to couple to a fitting through friction, compression, threading, adhesive, and the like. In some cases, the bottom wall 115 can defines a set of threads and grooves that are configured to couple to corresponding threads and grooves defined by a medical port. In some cases, the bottom wall 115 can define a structure for providing haptic feedback to a user for coupling to a female connector. For example, the bottom wall 115 can further define a ridge or lip configured to couple to a corresponding lip or ridge defined by a medical port. The structure can create an audible click or other noise, or a haptic reaction when the ridge or lip of the bottom wall 115 contacts or passes over the lip or ridge of the medical port. In some other cases, the female coupling adapter can be formed with the corresponding female connector. For example, the female coupling adapter can be integrated with a female connector via a molding process, through adhesive bonding, through ultrasonic welding, and the like.

The sidewalls 105 can also define a set of perforations 120. The perforations 120 can in some cases have a length offset from the length of the sidewalls 105. For example, the length of the perforations can be diagonal to the length of the sidewalls (as shown in FIG. 1, where the length of the sidewalls is defined as the distance between the top and bottom ends of the adapter). In other cases, the length of the perforations 120 can be parallel to, or perpendicular to, the length of the sidewalls 105. The perforations 120 can provide enhanced compressibility characteristics, for example, when coupling the adapter to a medical port. In some cases, the perforations 120, or a portion thereof, can be covered by a thin sheet of material (e.g., either exterior to or within the compressible lumen formed by the sidewalls 105. The thin sheet of material may prevent, reduce, or mitigate liquid flow through the perforations 120. The thin sheet can be molded integrally with the sidewalls 105.

### Male Coupling Adapter

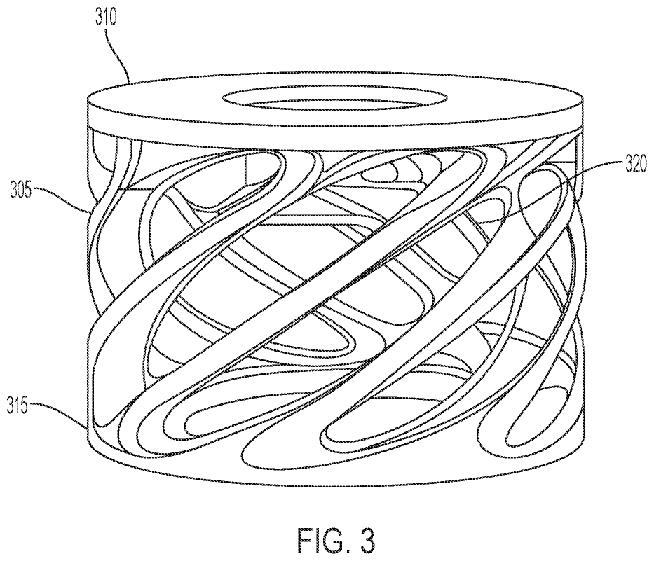

FIG. 3 depicts a male coupling adapter or contamination guard, according to an embodiment of the claimed invention. The adapter can include a hollow structure configured for encompassing a male connector or medical port. The adapter can include a set of sidewalls (e.g., a medial region) 305. In some cases, the adapter can be of a cylindrical shape, but the adapter can be other shapes as well (rectangle tube, helix, and the like). The sidewalls 305 can couple to a top wall (e.g., a distal collar) 310 on one end of the adapter. The top wall 310 can define a top orifice for accommodating a coupling between a male connector to a corresponding female connector. The top wall 310 can also be configured to couple to a ridge or top wall of a corresponding female coupling adapter described in more detail above (e.g., ridge 110 of FIG. 1). For example, a width of the top wall 310 can be approximately equivalent to an interior width defined by the ridge of a corresponding female coupling adapter.

The sidewalls 305 can also couple to a bottom wall (e.g., a proximal collar) 315 on another end of the adapter. The bottom wall 315 can define an orifice, which can be configured for accommodating the disposition of a male connector. The male coupling adapter can be configured for housing or accommodating different male connectors of varying widths and/or lengths, including universal male luer lock connectors. For example, the bottom wall 315 can be adapted and configured to couple to a fitting through friction, compression, threading, adhesive, and the like. In some cases, the bottom wall 315 can define a set of threads and grooves that are configured to couple to corresponding threads and grooves defined by a medical port. In some cases, the bottom wall 315 can define a structure for providing haptic feedback to a user for coupling to a female connector. For example, the bottom wall 315 can further define a ridge or lip configured to couple to a corresponding lip or ridge defined by a medical port. The structure can create an audible click or other noise, or a haptic reaction when the ridge or lip of the bottom wall 315 contacts or passes over the lip or ridge of the medical port. In some other cases, the male coupling adapter can be formed with the corresponding male connector. For example, the male coupling adapter can be integrated with a male connector via a molding process, through adhesive bonding, through ultrasonic welding, and the like.

The sidewalls 305 can also define a set of perforations 320. The perforations 320 can in some cases have a length offset from the length of the sidewalls 305. For example, the length of the perforations 320 can be diagonal to the length of the sidewalls 305 (as shown in FIG. 3). The perforations 320 can provide enhanced compressibility characteristics, for example, when coupling the adapter to a medical port. In some cases, the perforations 320, or a portion thereof, can be covered by a thin sheet of material (e.g., either exterior to or within the compressible lumen formed by the sidewalls 305. The thin sheet of material may mitigate liquid flow through the perforations 320.

### Removable Seal

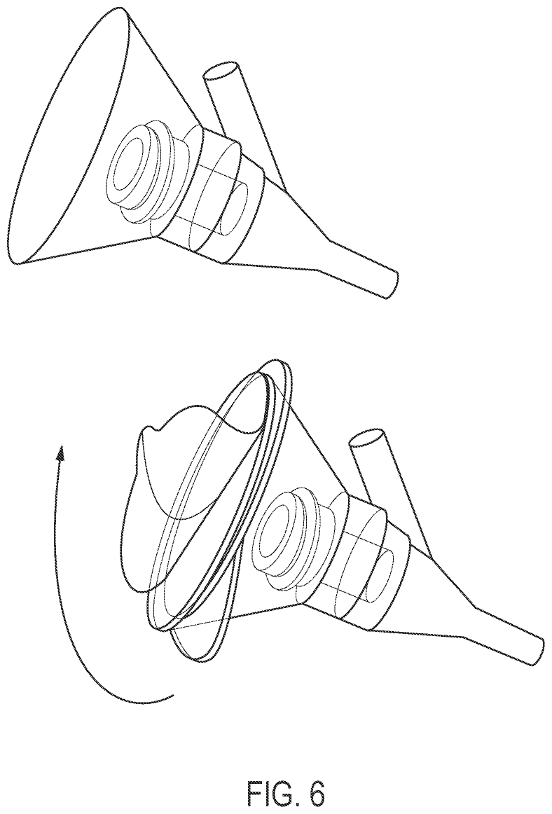

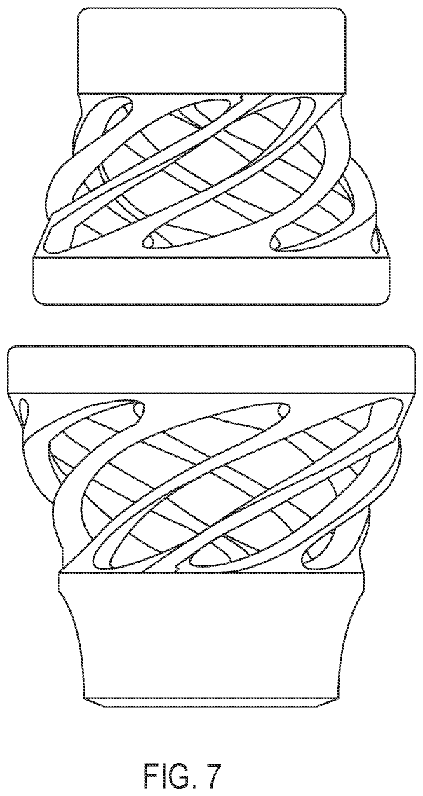

In some cases, a covering seal 605 (as shown in FIG. 6) can be detachably disposed on the ridge 110 and/or top wall 310, such that the top orifice is covered (not exposed to the environment) by the covering seal 605. The covering seal 605 can be composed of a thin plastic, paper, foil, and the like, which can be peeled off the ridge 110 and/or top wall 310 prior to using the coupling adapter or contamination guard. The covering seal 605 can be coupled to the ridge 110 and/or top wall 310 with a temporary adhesive, static friction (e.g., the covering seal 605 is compressed onto the ridge 110), and the like. Further, in some cases the covering seal 605 can define a tab that extends away from the ridge 110 and/or top wall 310, which can provide a point for a user to grip the covering seal 605 for seal removal.

### Interaction Between Adapters

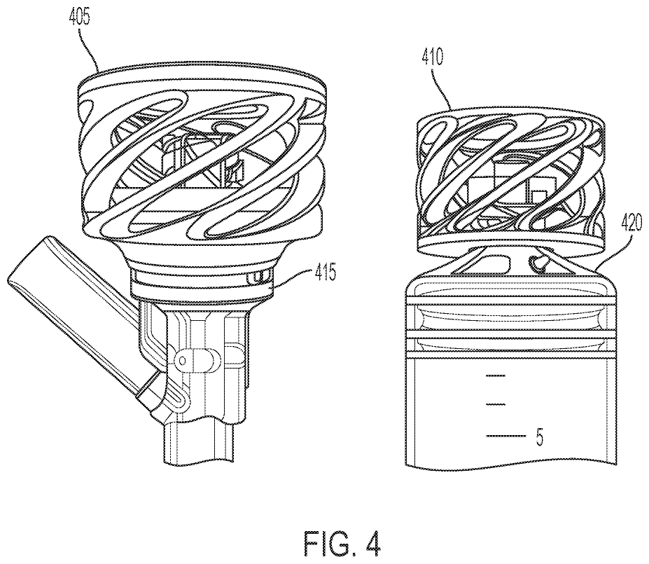

FIG. 4 depicts a female coupling adapter 405 and a male coupling adapter 410 according to embodiments of the claimed invention. As shown, the female coupling adapter 405 is coupled to a female connector 415 (e.g., on an intravenous line), and the male coupling adapter 410 is coupled to a male connector 420 (e.g., on a syringe). The female connector 415 can be disposed within the female coupling adapter 405 through the orifice defined by the top wall of the female coupling adapter 405. Likewise, the male connector 420 can be disposed within the male coupling adapter 410 through the orifice defined by the top wall of the male coupling adapter 410. As shown, the ends of the connectors 415 and 420 can be disposed within the corresponding cavity of the coupling adapters 405 and 410, which can prevent or mitigate physical contamination of the connectors 415, 420.

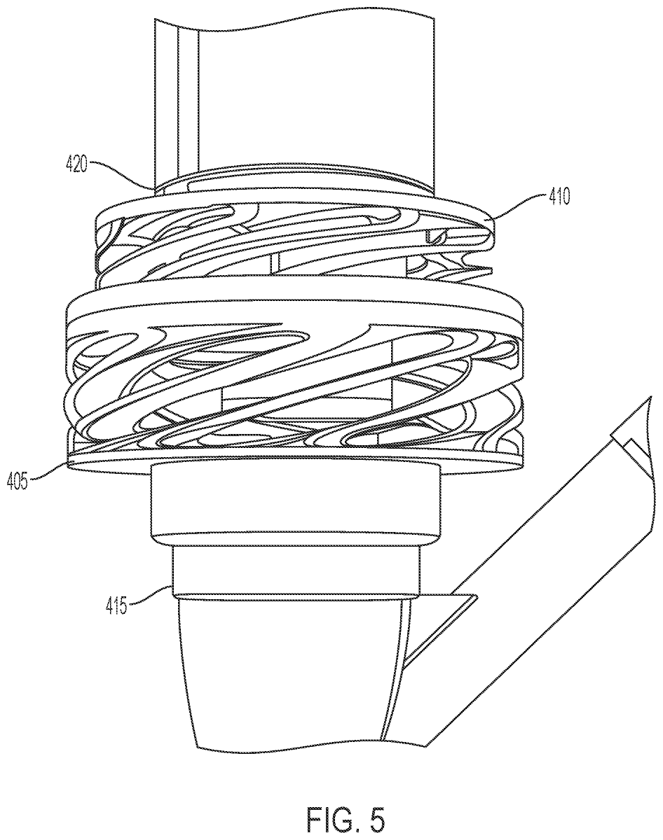

FIG. 5 depicts a female coupling adapter 405 and a male coupling adapter 410 according to embodiments of the claimed invention. Similar to FIG. 4, the female coupling adapter 405 is coupled to a female connector 415, and the male coupling adapter 410 is coupled to a male connector 420. The female connector 415 can also be coupled to the male connector 420, such as inserting the male connector 420 into the female connector 415. As the male connector 420 is inserted into the female connector 415, the male connector 420 can enter into the cavity of the female coupling adapter 405 via the orifice defined by the top wall of the female coupling adapter 405. Further, the top wall of the male coupling adapter 410 can come in contact with the top wall and/or the ridge defined by the sidewalls of the female coupling adapter 405. The male and female connectors 415 and 420 can couple to one another by applying a compression force to the connectors 415 and 420. This locking of the connectors can in turn compress the male coupling adapter 410, the female coupling adapter 405, or both, along the sidewall lengths of the corresponding adapter(s). The compression of the adapters is shown in FIG. 5, where the diagonal perforations defined by the sidewalls of the respective adapter provide for greater elasticity in the respective sidewall. Further, as can be seen in FIG. 5, not only are the individual male and female connectors 415 and 420 are encompassed by the corresponding coupling adapter, but the connection between the connectors is encompassed as well. When the compressive force is removed from the coupling adapters, for example by uncoupling the adapters, or in some cases when the coupling adapters are locked to each other (e.g., such that a compressive force is no longer needed), the coupling adapters can revert to their previous dimensions (e.g., without manual manipulation for the reversion), which can occur due in part to the elastic composition of the adapter s well as the structure of the adapters (e.g., the number and shape of the legs forming the perforations).

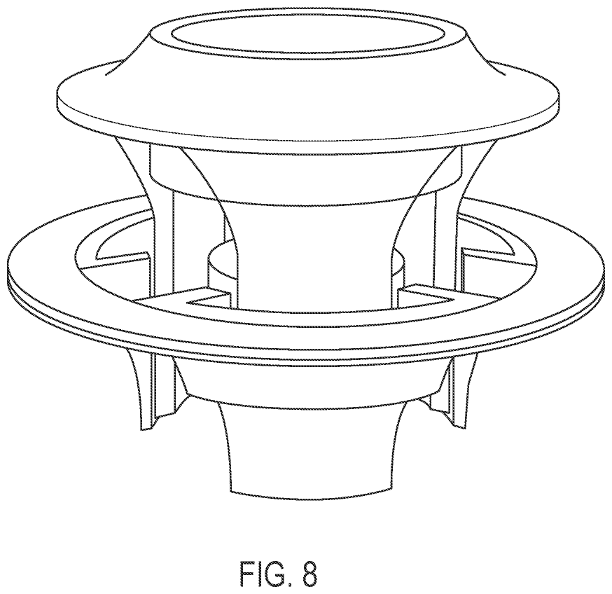

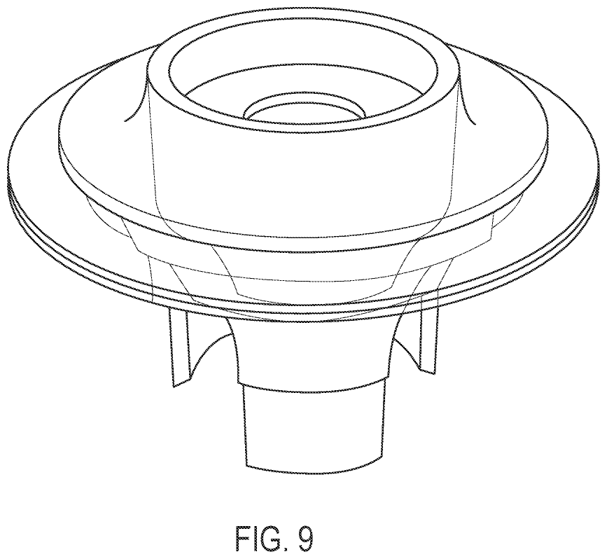

In some cases the corresponding adapters can include flanges or protrusions on the respective distal collars. FIGS. 8 and 9 depict adapter with flanges along extending away from the distal collar. These flanges can increase contacted surface areas between the adapters when coupled to one another. The flanges can mitigate potential slippage between the adapters when compressed, and can further increase the changes of a proper coupling between the adapters.

### Exemplary Materials

The devices described herein can be formed from a thermoplastic material and/or an elastomer. Exemplary elastomers include Methyl Methacrylate Acrylonitrile Butadiene Styrene (MABS), rubber, latex, polyvinyl chloride (PVC), nitrile rubber, neoprene, isoprene, artificial polyisoprene, polyurethane, and the like. Elasticity can, alone or in combination with the perforations 120 in the sidewalls 105, allow the adapter to compress when a force is received from either the bottom wall 115 and/or the ridge 110.

In some cases, the coupling adapter can include compositions that have antimicrobial properties. For example, the coupling adapters can include additives such as silver. In some cases, a coating can be applied to the external surfaces of a coupling adapter that can include antimicrobial properties. For example, a coating can be a nano-coating that is fluid-resistant, which can in some cases also provide protection from corrosion and/or mechanical resistance. In some cases, the coating can be an antibiotic coating, such as a polymer coating that can be used in controlled-release drugs. In some other cases, the coating can be a biosurfactant. In some cases, the coating can be fluorine, which can provide a hydrophobic surface. In some cases, the coating can be a metal coating (e.g., silver, copper, and the like).

In some cases, the coupling adapters can include compositions that provide an indication of a characteristic of the coupling adapter. For example, the coupling adapter can include a thermochromic dye (e.g., either within the coupling adapter composition or as a coating), which can change color in response to a temperature change, such as when a cleaning product (e.g., isopropyl alcohol) is placed on the surface and allowed to evaporate, causing a quick temperature change.

In some cases, a coating can include an indicator for pH change. For example, a universal pH indicator can be coated on the exterior surface of the adapter, which can be composed of 1-propanol, phenolphthalein, sodium hydroxide, methyl red, bromothymol blue, sodium bisulfate, thymol blue, beet extract, turnip extract, and the like.

In some cases, the coupling adapter can include an electrical resistance indicator. For example, a coating, or the adapter composition, can include silicon and gold sensor(s) that can shift current output based on temperature, and/or can increase temperature based on current value.

In some cases, the coupling adapter can include a fermentation coating, such as a mannitol salt coating, that can change color based on growth of certain bacteria (e.g., staph aureus).

In some cases, the adapter can include a mechanical stress indicator, such as a mechanochromic or piezochromic material, that can change color when a mechanical stimulus or stress is experienced by the adapter.

In some cases, the adapter can include a pathogen indicator, such as a biochromic material or a biochromic conjugated polymer (BCP).

In some cases, the adapter can include a photochromic coating, which can for example change color with changing light intensity (e.g., for indicating proper UV sterilization).

Exemplary polymer compositions that can be a part of an adapter composition, or can be coated onto the adapter surface(s) include: chitosan, chitosan-based coatings functionalized with methacrylate-based polymer brushes, polypyrrole/chitosan composites, gelatin, gelatin/chlorhexidine acetate (CHA), cellulose, dextran, cyclodextrin, 2-(4-methylthiazol-5-yl) ethyl methacrylate (MTA) and N-(3,4-dihydroxyphenethyl) methacrylamide (DOMA) copolymers, poly(dimethylaminoethylmethacrylate)-functionalized graphene oxide (GO-QPDMAEMA), N-(3,4-dihydroxyphenethyl) methacrylamide (EXDMA) and 2-(4-methylthiazol-5-yl) ethyl methacrylate (MTA) quaternized with methyl iodide PEG brush surfaces-PLL coils composites and cationically functionalized gold nanoparticles, poly-L-lysine (PEL) and hyaluronic acid (HA) denoted PLL30, PLL90, and PLL400, cationic acrylate-based copolymers (PAMs) by 3-(methacryloylamino) propyl trimethyl ammonium chloride (MPAC) and acrylates (BA, MMA), multilayers of polyethylenimine (PEI) and styrene maleic anhydride copolymer (SMA), quaternized poly(4-vinylpyridine-co-N-vinylpyrrolidone) (P(4VP-co-NVP)) copolymers, Poly-arginine (PAR), PEI, PEI and PEI-based nanoparticles, and the like. This list is non-exhaustive, and one skilled in the art will understand that other compositions with similar characteristics can be used for the purposes described above.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A contamination guard comprising:
a proximal collar adapted and configured to engage with a medical port;
an elastomeric medial region coupled to the proximal collar; and
a distal collar coupled to the proximal collar by the elastomeric medial region;
wherein the elastomeric medial region is adapted and configured to:
bias the distal collar to extend at least partially over a distal end of the port when in an unloaded position; and
allow the distal collar to resiliently travel proximally when loaded to permit engagement with the distal end of the medical port.

2. The contamination guard of claim 1, wherein the proximal collar and the distal collar are annular.

3. The contamination guard of claim 1, wherein the elastomeric medical region comprise a plurality of legs.

4. The contamination guard of claim 3, wherein the plurality of legs are axial, trussed, or helical.

5. The contamination guard of claim 1, wherein the distal collar is elastomeric.

6. The contamination guard of claim 1, wherein the elastomeric medial region is fabricated from the group consisting of: silicone and Methyl Methacrylate Acrylonitrile Butadiene Styrene (MABS).

7. The contamination guard of claim 1, further comprising a covering seal adapted and configured to be detachably coupled to the distal collar and across an aperture defined by the distal collar.

8. The contamination guard of claim 1, wherein the proximal collar defines a set of threads and grooves, wherein the set of threads and grooves is adapted and configured to couple with another set of threads and grooves defined by medical port.

9. The contamination guard of claim 1, wherein the proximal collar is permanently coupled to the medical port.

10. The contamination guard of claim 1, further comprising:
an antimicrobial composition disposed on a surface of, or within a composition of, the proximal collar, the distal collar, the elastomeric medial region, or a combination thereof.

11. The contamination guard of claim 1, further comprising:
an indicator coating disposed on a surface of the proximal collar, the distal collar, the elastomeric medial region, or a combination thereof.

12. The contamination guard of claim 11, wherein the indicator coating provides an indication of bacterial contamination, a pH change, an oxidation or material breakdown, or a combination thereof.

13. The contamination guard of claim 1, further comprising a sheet of elastomeric material covering a surface of a portion of the proximal collar, the distal collar, the elastomeric medial region, or a combination thereof.

14. The contamination guard of claim 1, wherein the elastomeric medial region is further adapted and configured to allow the distal collar to travel distally to a neutral position when unloaded.

15. The contamination guard of claim 1, wherein the distal end defines a set of ridges or flanges extending outwardly away from the distal end.
